(19) United States
(12) Patent Application Publication
(10) Pub. No.: US 2002/0006394 A1
(43) Pub. Date: Jan. 17, 2002

(54) Photochemical tissue bonding

(76) Inventors: Robert W. Redmond, West Newton, MA (US); Barbara Chan, Boston, MA (US)

Correspondence Address: Thomas J. Kowalski, FROMMER LAWRENCE & HAUG LLP, 745 Fifth Avenue, New York, NY, 10151, US

(21) Appl. No.: 09/900,504

(22) Filed: Jul. 6, 2001

Related U.S. Application Data
(63) Continuation-in-part of application No. 09/781,577, filed on Feb. 12, 2001.
(63) Continuation-in-part of application No. PCT/US01/40093, filed on Feb. 12, 2001.
(60) Provisional application No. 60/181,980, filed on Feb. 11, 2000.

(51) Int. Cl. A61K 45/00; A61N 1/30

(52) U.S. Cl. 424/93.7; 604/20

(57) ABSTRACT

Photochemical tissue bonding methods include the application of a photosensitizer to a tissue, e.g., cornea, followed by irradiation with electromagnetic energy to produce a tissue seal. The methods are useful for wound repair, or other tissue repair.

### CROSS-REFERENCE TO RELATED APPLICATIONS

This is a continuation-in-part application of U.S. application Ser. No. 09/781,577, filed Feb. 12, 2001, claiming priority to U.S. Provisional Application Serial No. 60/181,980, filed Feb. 11, 2000, the contents of which are incorporated herein by reference. Reference is also made to PCT application No. PCT/US01/40093, filed on Feb. 12, 2001, claiming priority to U.S. Provisional Application Serial No. 60/181,980, filed Feb. 11, 2000, the contents of which are incorporated herein by reference.

### BACKGROUND

Traditional wound closure methods, such as staples and sutures, have numerous drawbacks, including the possible occurrence of inflammation, irritation, infection, wound gape, and leakage. The cosmetic results of the use of staples and sutures can also be undesirable. In corneal applications, sutures often produce astigmatism due to uneven suture tension. In skin grafting techniques, sutures can lead to a variety of complications in wound healing, including foreign body responses that cause scarring. Traditional wound closure methods suffer from a number of drawbacks that are overcome by the present invention.

Possible alternatives to sutures include hemostatic adhesives, such as fibrin sealants (Henrick et al. (1987) J Cataract Refract Surg 13:551-553; Henrick et al. (1991) J Cataract Refract Surg 17:551-555), cyanoacrylate adhesives (Shigemitsu et al. (1997) International Ophthalmology 20:323-328), and photodynamic tissue glue, composed of a mixture of riboflavin-5-phosphate and fibrinogen, which has been reported to close cataract incisions and attach donor cornea in corneal transplants (Goins et al. (1997) J Cataract Refract Surg 23:1331-1338; Goins et al. (1998) J Cataract Refract Surg 24:1566-1570; U.S. Pat. No. 5,552,452). In addition, temperature-controlled tissue welding has been attempted in bovine cornea and rat intestine (Barak et al. (1997) Surv Ophthalmol 42 Supp.1:S77-81; Cilesiz et al. (1997) Lasers Surg Med 21:269-86). Photochemical tissue welding of dura mater has also been reported, using 1,8 naphthalimides irradiated with visible light (Judy et al. (1993) Proc. SPIE - Int. Soc. Opt. Eng. 1876:175-179).

Skin grafts and/or skin substitutes (need discussion for next disclosure) are widely used in surgical procedures such as skin transplantation, burn and ulcer wound management and plastic surgery. Current fixation aids for grafting mainly consist of mechanical and adhesive means (Bass & Treat (1995) Lasers Surg Med 17:315-49). Surgical sutures and staples mechanically hold the tissue in position while tissue and fibrin glues chemically/biochemically bond the graft to the host. However, the use of sutures and staples has low aesthetic/cosmetic value and may lead to foreign-body reactions as well as wound complications (Bass & Treat (1995) Lasers Surg Med 17:315-49). The use of tissue glues such as cyanoacrylate provides excellent binding strength but results in persistent inflammation and foreign body giant cell reaction (Forseth D M et al. (1992) J Long Term Eff Med Implants 2(4):221-33, Toriumi D M et al. (1990) Arch Otololaryngol Head Neck Surg 116:546-550). Although the use of autogenous fibrin glue eliminates the foreign-body reactions and the associated complications, it elicits other problems. Firstly, it is costly and time-consuming to extract and purify autogenous fibrinogen from the patient's blood (Dahlstrom K K et al. (1991) Skin Transplantation 89(5):968-72) and secondly, the mechanical outcome is not satisfactory since the breaking strength at the interface was less than 0.2N/cm² (Dahlstrom K K et al. (1991) Skin Transplantation 89(5):968-72).

The ideal technique for wound closure would be simpler, more rapid, and prone to fewer post-operative complications than conventional techniques. In the cornea, an ideal tissue repair or wound closure technique would produce a watertight seal without inducing astigmatism. In skin grafting, techniques enabling rapid and sustained adherence to the wound surface and the ability to resist shear stress are ideal for successful graft take.

### SUMMARY

The present invention is based, in part, on the discovery that the application of a photosensitizer, e.g., Rose Bengal (RB), riboflavin-5-phosphate (R-5-P), methylene blue (MB), or N-hydroxypyridine-2-(1H)-thione (N-HTP), to a tissue, e.g., cornea, skin, tendon, cartilage, or bone, followed by photoactivation, e.g., irradiation with electromagnetic energy, e.g., light, can produce a tissue-tissue seal (e.g., to repair a wound, or seal a tissue transplant) without collagen denaturation or heat induced peripheral tissue damage. Furthermore, the tissue-tissue seal can be produced when the photosensitizer is applied to the tissue in the absence of an exogenously supplied source of cross-linkable substrate, e.g., protein, e.g., fibrin or fibrinogen, or protein-based tissue adhesive or glue. Such exogenous substances are often suggested to be used to contribute cross-linkable protein to a tissue. (Herein, a graft tissue or the components thereof is not considered such a source of exogenously supplied cross-linkable substrate.) This procedure is referred to herein as photochemical tissue bonding (PTB). PTB can be used ex vivo or in vivo in a subject, e.g., a human, or a non-human animal.

Accordingly, in one aspect, the invention features, a method for cross-linking tissue, e.g., creating a tissue seal, such as in tissue grafting. The method includes identifying a tissue in need of repair, e.g., a collagenous tissue, e.g., cornea, skin, bone, cartilage, or tendon; staining the tissue with a photosensitizer, and optionally a second tissue, e.g. a tissue graft, with at least one photosensitizer agent, e.g., Rose Bengal (RB), riboflavin-5-phosphate (R-5-P), methylene blue (MB), or N-hydroxypyridine-2-(1H)-thione (N-HTP), to form a photosensitizer-stained tissue complex; and applying electromagnetic energy, e.g., light, to the tissue-photosensitizer association sufficient to produce cross linking of a protein, e.g., collagen, in the tissue. The tissue is not contacted with an exogenously supplied source of cross-linkable substrate, e.g., protein, e.g., fibrin or fibrinogen, or protein-based tissue adhesive or glue, which is cross linked by the application of electromagnetic energy. (Herein, a graft tissue or the components thereof is not considered such a source of exogenously supplied cross-linkable substrate.)

In a preferred embodiment, the tissue is corneal tissue. E.g., one or more elements, e.g., cut or otherwise separated edges or surfaces, of the subject's corneal tissue can be joined together, or to graft tissue.

In a preferred embodiment, the tissue is in need of repair. For example, the tissue, e.g., cornea, has been subjected to trauma, a surgical incision, LASIK flap reattachment, corneal transplant, or correction of astigmatism. This tissue can be of any type where wound closure is necessary, for example a cardiovascular, neurological, gastrointestinal, urological, occular or musculoskeletal (including orthopedic and dermal) tissue. Wound closure can comprise the joining of cut or otherwise separated edges or surfaces of the tissue/damaged tissue.

In a preferred embodiment, the tissue in need of repair is grafted with an exogenous tissue. An exogenous tissue is one supplied from a site other than the site of the lesion/wound. Preferably, this tissue is skin. This tissue can be of any type where wound closure is necessary, for example a cardiovascular, neurological, gastrointestinal, urological, occular or musculoskeletal (including orthopedic and dermal) tissue.

In a preferred embodiment, the photosensitizer agent is selected from the group consisting of Rose Bengal, riboflavin-5-phosphate, methylene blue, and N-hydroxypyridine-2-(1H)-thione.

In a preferred embodiment, the photosensitizer agent is Rose Bengal.

In a preferred embodiment, the contacting step occurs ex vivo.

In a preferred embodiment, the contacting step occurs in vivo in a subject, e.g., a human, or an non-human animal, preferably a non-albino animal, e.g., a non-albino rabbit.

In a preferred embodiment, the subject is other than an albino animal, e.g., other than an albino rabbit.

In a preferred embodiment, the subject is a human.

In a preferred embodiment, the application of electromagnetic energy to the tissue-photosensitizer complex occurs without substantial thermal tissue damage, e.g., shrinkage or deformation around the wound site and thermal cell damage.

In a preferred embodiment, the application of electromagnetic energy to the tissue-photosensitizer complex occurs without more than a 15° C. rise in temperature as measured, e.g., with an imaging thermal camera during irradiation.

In a preferred embodiment, the application of electromagnetic energy to the tissue-photosensitizer complex occurs without more than a 10° C. rise in temperature as measured, e.g., with an imaging thermal camera during irradiation.

In a preferred embodiment, the application of electromagnetic energy to the tissue-photosensitizer complex occurs without more than a 3° C. rise in temperature as measured, e.g., with an imaging thermal camera during irradiation.

In a preferred embodiment, the application of electromagnetic energy to the tissue-photosensitizer complex occurs without more than a 2° C. rise in temperature as measured, e.g., with an imaging thermal camera during irradiation.

In a preferred embodiment, the application of electromagnetic energy to the tissue-photosensitizer complex occurs without more than a 1° C. rise in temperature as measured, e.g., during irradiation with an imaging thermal camera.

In another aspect, the invention features, a method for repairing a corneal lesion, e.g., a corneal incision, laceration, or a corneal transplant, in a subject, e.g., a human, or a non-human animal, preferably a non-albino animal. The method includes: contacting a corneal tissue with at least one photosensitizer agent, e.g., RB, R-5-P, MB, or N-HTP, and applying electromagnetic energy, e.g., light, to the corneal tissue-photosensitizer complex sufficient to produce a reactive species, e.g., a reactive oxygen species, from the photosensitizer. The corneal tissue is not contacted with an exogenously supplied source of cross-linkable substrate, e.g., protein, e.g., fibrin or fibrinogen, or protein-based tissue adhesive or glue, which is cross-linked by the application of electromagnetic energy.

In a preferred embodiment, the corneal lesion is caused by a surgical procedure.

In a preferred embodiment, the surgical procedure is selected from the group consisting of corneal transplant surgery, cataract surgery, laser surgery, keratoplasty, penetrating keratoplasty, posterior lamellar keratoplasty, LASIK, refractive surgery, cornea reshaping, and treatment of corneal laceration.

In a preferred embodiment one or more elements, e.g., cut or otherwise separated edges or surfaces, of the subject's corneal tissue can be joined together, or to graft tissue.

In a preferred embodiment, a subject's muscle tendon can be joined to the subject's eye. E.g., an ocular misalignment can be reduced, adjusted, or corrected, e.g., by joining an eye muscle tendon to the eye.

In another preferred embodiment, the cornea is in need of correction for astigmatism. For example, PTB can be used to correct, reduce, or decrease astigmatism, e.g., by inducing astigmatism in the orthogonal meridian, thereby counteracting preexisting astigmatism. In a preferred embodiment, PTB induces a predictable degree of corrective astigmatism.

In a preferred embodiment, the method further comprises administration of an adjunctive therapy, e.g., contact lens therapy, amniotic membrane therapy, LASIK therapy, or administration of antibiotics.

In a preferred embodiment, the electromagnetic energy applied is greater than 1200 J/cm². In another preferred embodiment, the electromagnetic energy applied is between 200 and 1200 J/cm². In another preferred embodiment, the electromagnetic energy applied is between 200 and 800 J/cm². In yet another preferred embodiment, the electromagnetic energy applied is between 200 and 500 J/cm². In yet another preferred embodiment, the electromagnetic energy applied is between 300 and 600 J/cm². In another preferred embodiment, the electromagnetic energy applied is between 350 and 550 J/cm².

In a preferred embodiment, the electromagnetic energy is applied at an irradiance less than 3.5 W/cm².

In a preferred embodiment, the electromagnetic energy is applied at an irradiance less than 1.5 W/cm².

In a preferred embodiment, the electromagnetic energy is applied at an irradiance of about 0.60 W/cm².

In a preferred embodiment, the subject is other than an albino animal, e.g., other than an albino rabbit.

In another aspect, the invention features, a method for repairing a corneal lesion in vivo in a living subject, e.g., a human, or a non-human animal, preferably a non-albino animal. The method includes contacting a corneal tissue with Rose Bengal (RB) to form a corneal tissue-RB complex; and applying electromagnetic energy, e.g., light, to the corneal tissue-RB complex in a manner effective to elicit the production of a reactive species, e.g., a reactive oxygen species, from the RB. The corneal tissue is not contacted with an exogenously supplied source of cross-linkable substrate, e.g., protein, e.g., fibrin or fibrinogen, or protein-based tissue adhesive or glue, which is cross-linked by the application of electromagnetic energy.

In a preferred embodiment, the subject is a human.

In a preferred embodiment, the corneal lesion is caused by a surgical procedure.

In a preferred embodiment, the surgical procedure is selected from the group consisting of corneal transplant surgery, cataract surgery, laser surgery, keratoplasty, penetrating keratoplasty, posterior lamellar keratoplasty, LASIK, refractive surgery, cornea reshaping, and treatment of corneal laceration.

In a preferred embodiment one or more elements, e.g., cut or otherwise separated edges or surfaces, of the subject's corneal tissue can be joined together, or to graft tissue.

In a preferred embodiment, a subject's muscle tendon can be joined to the subject's eye. E.g., an ocular misalignment can be reduced, adjusted, or corrected, e.g., by joining an eye muscle tendon to the eye.

In another preferred embodiment, the cornea is in need of correction for astigmatism. For example, PTB can be used to correct, reduce, or decrease astigmatism, e.g., by inducing astigmatism in the orthogonal meridian, thereby counteracting preexisting astigmatism. In a preferred embodiment, PTB induces a predictable degree of corrective astigmatism.

In a preferred embodiment, the method further comprises administration of an adjunctive therapy, e.g., contact lens therapy, amniotic membrane therapy, LASIK therapy, or administration of antibiotics.

In a preferred embodiment, the subject is other than an albino animal, e.g., other than an albino rabbit.

In another aspect, the invention features a kit for repairing corneal lesions, which kit includes a photosensitizer agent, e.g., RB, R-5-P, MB, or N-HTP, instructions for photoactivation of the photosensitizer agent to repair the corneal lesion, accessory tools and instructions for effective tissue edge approximation. In a preferred embodiment the kit does not include a source of cross-linkable substrate, e.g., protein, e.g., fibrin or fibrinogen, or protein-based tissue adhesive or glue, for use with the photosensitizer.

In a preferred embodiment, the photosensitizer agent is Rose Bengal.

The details of one or more embodiments of the invention are set forth in the accompanying drawings and the description below.

Other features, objects, and advantages of the invention will be apparent from the description and drawings, and from the claims.

### DESCRIPTION OF THE DRAWINGS

FIG. 1. Typical trace of increasing IOP with infusion time for a PTB treated eye showing IOPL at 300 mm Hg.

FIG. 2. Mean IOPL values for PTB treated eyes (n=5) using 514 nm light (2.55 W/cm²) and RB (1.5 mM) in PBS. Additional controls are incisions treated with RB or buffer but no laser light.

FIG. 3. Mean IOPL before and after PTB using RB and 514 nm irradiation. RB (10 μl, 1.5 mM) was applied to the incision surfaces then treated with the doses indicated using irradiances of: (A) 1.27 W/ cm2, (B) 2.55 W/cm2 and (C) 3.82 W/cm2.

FIG. 4. Mean IOPL before and after PTB using R-5-P and 488 nm irradiation. R-5-P (40 μl, 11 mM) was applied to the incision surfaces then treated with the doses indicated using irradiances of: (A) 1.27 W/cm2, (B) 2.55 W/cm2 and (C) 3.82 W/cm2.

FIG. 5. Mean IOPL values before and after PTB using Fl and 488 nm irradiation. Fl (40 μl, 0.6 mM) was applied to the incision surfaces then treated with the doses indicated using irradiances of: (A) 1.27 W/cm2, (B) 2.55 W/cm2 and (C) 3.82 W/cm2.

FIG. 6. A tensiometer coupled to a force transducer. Force was applied along the direction of the skin grafts at a constant speed of 12.7 mm/min by pulling on the pre-attached suture loop.

FIG. 7. Association between dermal uptake and time of RB exposure. An increase in exposure time to 10 minutes increased the depth of dermal uptake to approximately 25 microns.

FIG. 8. Energy needed to separate skin grafts. Use of RB at a concentration of 0.1% (w/v) provided an optimal increase in adherence levels.

FIG. 9. Energy needed to separate skin grafts after PTB at 0.56 and 1.68 W/cm². Irradiation levels of 0.56 and 1.68 W/cm² provided a positive dose-dependent relationship between the laser energy (fluence) and the adherence of the skin grafts.

FIG. 10. Cell viability and collagen organization. Following irradiation levels of both 0 (control) and 0.56 W/cm², skin grafts were viable, as indicated by the dark blue precipitates in the cytoplasm contrasted by the red nuclear counterstain.

### DETAILED DESCRIPTION

Photochemical tissue bonding (PTB), as described herein, provides a method to create a tissue-tissue seal, e.g., to treat a wound, e.g., a corneal wound, without collagen denaturation or heat-induced peripheral tissue damage. PTB, as described herein, involves the application of a photosensitizer to a wound surface followed by photoactivation by laser irradiation to seal the wound. The photosensitizer can be effectively applied to seal a wound, or otherwise repair a tissue, such as by graft, in the absence of an exogenous protein-based adhesive, such as fibrinogen.

Methods of the invention provide strong covalent bonding at the tissue to tissue interface and have no requirement for an exogenous protein (other than what may be present in a tissue graft), e.g., fibrinogen, that must be isolated from the patient to be treated or derived from one or more donors. Methods of the invention do not require the use of chemical glues, e.g., cyanoacrylate adhesives and surgical sutures. The methods described herein minimize tissue thermal denaturation of proteins caused by tissue heating.

Current methods of skin grafting are complicated by multiple use of sutures, low cosmetic value, wound complications such as foreign body reactions, void and non-adherent grafts. The present invention overcomes problems known in the art. The methods described herein are ideal for tissues in need of repair and/or a water-tight seal. These tissues can be of any type where wound closure is necessary, for example a cardiovascular, neurological, gastrointestinal, urological, occular or musculoskeletal (including orthopedic and dermal) tissue. Wound closure can comprise the joining of cut or otherwise separated edges or surfaces of the damaged tissue. Wound closure can further comprise the grafting of an exogenous tissue on to the surface of a damaged tissue. Preferably, this tissue is skin. This tissue can be of any type where wound closure is necessary, for example a cardiovascular, neurological, gastrointestinal, urological, occular or musculoskeletal (including orthopedic and dermal) tissue.

Closure of corneal wounds or corneal transplants with sutures can be associated with neo-vascularisation, rejection of the donor cornea, and induced post-operative astigmatism partly due to uneven suture tension. This can occur after penetrating keratoplasty where numerous sutures are needed to hold the graft in place. Suturing techniques designed to evenly distribute tension across corneal grafts may still result in significant astigmatism. Additionally, loose or broken sutures can leave a patient vulnerable to microbial keratitis. The sutures used are skill intensive and are mainly performed by corneal specialists. The methods described herein do not require the use of sutures. Although factors such as wound healing, host graft sizing and trephination techniques also play a role in post-operative astigmatism, the methods described herein hold the graft with equally distributed force and help reduce post-operative astigmatism. PTB reduces the operating and rehabilitation time for procedures to close wounds, e.g., to treat incisions or corneal lacerations, spot seal LASIK flaps, perform cataract surgery, and attach donor cornea.

### Photoactivation and Photosensitizers

The methods to create a tissue-tissue seal described herein include treating a tissue with a photosensitizer agent, e.g., RB, R-5-P, MB, or N-HTP, preferably in the absence of an exogenous protein, e.g., a protein based adhesive, e.g., fibrin or fibrinogen, and photoactivating the photosensitizer agent with electromagnetic radiation, e.g., light.

Photoactivation is used to describe the process by which energy in the form of electromagnetic radiation is absorbed by a compound, e.g., a photosensitizer, thus “exciting” the compound, which then becomes capable of converting the energy to another form of energy, preferably chemical energy. The electromagnetic radiation can include energy, e.g., light, having a wavelength in the visible range or portion of the electromagnetic spectrum, or the ultra violet and infra red regions of the spectrum. The chemical energy can be in the form of a reactive species, e.g., a reactive oxygen species, e.g., a singlet oxygen, superoxide anion, hydroxyl radical, the excited state of the photosensitizer, photosensitizer free radical or substrate free radical species. The photoactivation process described herein preferably involves insubstantial transfer of the absorbed energy into heat energy. Preferably, photoactivation occurs with a rise in temperature of less than 15 degrees Celsius (C.), more preferably a rise of less than 10 degrees C., more preferably a rise of less than 3 degrees C., more preferably a rise of less than 2 degrees C. and even more preferably, a rise in temperature of less than 1 degree C. as measured, e.g., by an imaging thermal camera that looks at the tissue during irradiation. The camera can be focused in the area of original dye deposit, e.g., the wound area, or on an area immediately adjacent the wound area, to which dye will diffuse. As used herein, a “photosensitizer” is a chemical compound that produces a biological effect upon photoactivation or a biological precursor of a compound that produces a biological effect upon photoactivation. Preferred photosensitizers are those that absorb electromagnetic energy, such as light. While not wishing to be bound by theory, the photosensitizer may act by producing an excited photosensitizer or derived species that interacts with tissue, e.g., collagenous tissue, to form a bond, e.g., a covalent bond or crosslink. Photosensitizers typically have chemical structures that include multiple conjugated rings that allow for light absorption and photoactivation. Examples of photosensitive compounds include various light-sensitive dyes and biological molecules such as, for example, xanthenes, e.g., rose bengal and erythrosin; flavins, e.g., riboflavin; thiazines, e.g., methylene blue; porphyrins and expanded porphyrins, e.g., protoporphyrin I through protoporphyrin IX, coproporphyrins, uroporphyrins, mesoporphyrins, hematoporphyrins and sapphyrins; chlorophylis, e.g., bacteriochlorophyll A, and photosensitive derivatives thereof. Preferred photosensitizers for use in the methods described herein are compounds capable of causing a photochemical reaction capable of producing a reactive intermediate when exposed to light, and which do not release a substantial amount of heat energy. Preferred photosensitizers are also water soluble. Preferred photosensitizers include Rose Bengal (RB); riboflavin-5-phosphate (R-5-P); methylene blue (MB); and N-hydroxypyridine-2-(1H)-thione (N-HTP).

Without wanting to be bound by theory, it is believed that the chemical energy, e.g., a reactive oxygen species, produced by photoactivation of the photosensitizer agent with which the tissue to be repaired is contacted, binds and causes structural changes in the amino acids of the proteins of the tissue, resulting in the formation of covalent bonds, polymerization, or cross-links between amino acids of the tissue, thus creating a proteinaceous framework that serves to seal, repair, heal, or close the tissue lesion or wound. For example, as a result of PTB treatment, strong covalent cross-links are believed to form between collagen molecules on opposing surfaces of a corneal lesion to produce a tight tissue seal.

The photosensitizer agent, e.g., RB, R-5-P, MB, or N-HTP, can be dissolved in a biocompatible buffer or solution, e.g., saline solution, and used at a concentration of from about 0.1 mM to 10 mM, preferably from about 0.5 mM to 5 mM, more preferably from about 1 mM to 3 mM.

The photosensitizer agent can be administered to the tissue by, e.g., injection into the tissue, or application onto the surface of the tissue. An amount of photosensitizer sufficient to stain, e.g., to cover the walls of, the lesion or wound to be repaired, can be applied. For example, at least 10 Tl of photosensitizer solution, preferably 50 μl (microliter), 100 μl, 250 μl, 500 μl, or 1 ml, or more, of photosensitizer solution can be applied to a tissue, e.g., a cornea. Preferably, the photosensitizer has a binding efficiency, e.g., a collagen binding efficiency, such that the dye is predominantly bound to the surface of the incision.

The electromagnetic radiation, e.g., light, is applied to the tissue at an appropriate wavelength, energy, and duration, to cause the photosensitizer to undergo a reaction to affect the structure of the amino acids in the tissue, e.g., to cross-link a tissue protein, thereby creating a tissue seal. The wavelength of light can be chosen so that it corresponds to or encompasses the absorption of the photosensitizer, and reaches the area of the tissue that has been contacted with the photosensitizer, e.g., penetrates into the region where the photosensitizer presents. The electromagnetic radiation, e.g., light, necessary to achieve photoactivation of the photosensitizer agent can have a wavelength from about 350 nm to about 800 nm, preferably from about 400 to 700 nm and can be within the visible, infra red or near ultra violet spectra. The energy can be delivered at an irradiance of about between 0.5 and 5 W/cm², preferably between about 1 and 3 W/cm². The duration of irradiation can be sufficient to allow cross linking of one or more proteins of the tissue, e.g., of a tissue collagen. For example, in corneal tissue, the duration of irradiation can be from about 30 seconds to 30 minutes, preferably from about 1 to 5 minutes. The duration of irradiation can be substantially longer in a tissue where the light has to penetrate a scattering layer to reach the wound, e.g., skin or tendon. For example, the duration of irradiation to deliver the required dose to a skin or tendon wound can be at least between one minute and two hours, preferably between 10 and 30 minutes.

Suitable sources of electromagnetic energy include commercially available lasers, lamps, light emitting diodes, or other sources of electromagnetic radiation. Light radiation can be supplied in the form of a monochromatic laser beam, e.g., an argon laser beam or diode-pumped solid state laser beam. Light can also be supplied to a non-external surface tissue through an optical fiber device, e.g., the light can be delivered by optical fibers threaded through a small gauge hypodermic needle or an arthroscope. Light can also be transmitted by percutaneous instrumentation using optical fibers or cannulated waveguides.

The choice of energy source will generally be made in conjunction with the choice of photosensitizer employed in the method. For example, an argon laser is a preferred energy source suitable for use with RB or R-5-P because these dyes are optimally excited at wavelengths corresponding to the wavelength of the radiation emitted by the argon laser. Other suitable combinations of lasers and photosensitizers will be known to those of skill in the art. Tunable dye lasers can also be used with the methods described herein.

### Uses

The methods described herein are suitable for use in a variety of applications, including in vitro laboratory applications, ex vivo tissue treatments, but especially in in vivo surgical procedures on living subjects, e.g., humans, and non-surgical wound healing.

The methods described herein are particularly useful for surgical applications, e.g., to seal, close, or otherwise join, two or more portions of tissue, e.g., to perform a tissue transplant and/or grafting operation, or to heal damaged tissue, e.g., a corneal incision. The methods described herein can be used in surgical applications where precise adhesion is necessary, and/or where the application of sutures, staples, or protein sealants is inconvenient or undesirable. For example, in corneal transplants and other eye operations, surgical complications such as inflammation, irritation, infection, wound gape, leakage, and epithelial ingrowth, often arise from the use of sutures. The photochemical tissue binding methods described herein are particularly suitable for use in surgery or microsurgery, for example, in surgical operations or maneuvers of the eye, e.g., in the repair of corneal wounds or incisions, in refractive surgery (the correction of irregularities or defects in the cornea by “shaving” an even layer off the cornea), in keratoplasty, in corneal transplants, and in correction of astigmatism, e.g., by inducing astigmatism designed to counteract preexisting astigmatism, e.g., in the orthogonal meridian.

As another example, sutures cannot be satisfactorily used on bone joint cartilage because of their mechanical interference with the mutual sliding of cartilage surfaces required for joint motion. Neither can sutures be used to seal surfaces of small blood vessels with diameters 1-2 mm or less, as sutures impinge upon the vessel lumen, compromising blood flow. Further, in skin grafting, sutures can induce foreign body responses that lead to scarring. Thus, the methods described herein are also useful in surgical interventions of the small vascular tissue, joint cartilage, skin, gastro intestinal tract, nerve sheaths, small ducts (urethra, ureter, bile ducts, thoracic duct), oral tissue or even the inner ear. Other procedures where sutures or staples are not indicated or desirable, and where the photochemical tissue bonding methods described herein are useful, include procedures involving laparoscopic operations or interventions such as laparoscopic (LP) thoracic procedures, LP appendectomy, LP hernia repairs, LP tubal ligations and LP orbital surgeries.

The photochemical tissue bonding methods described herein can also be used in tissue grafting. In one embodiment, an exogenous tissue graft comprising tissue such as skin, muscle, vasculature, stomach, colon or intestine, can be placed over the surface of the wound, impregnated with the photosensitizer agent described herein, and photoactivated with a visible light source, e.g., an incandescent, fluorescent or mercury vapor light source, e.g., a xenon arc lamp, or a laser light source, e.g. argon-ion laser. Preferably, the photochemical bond enables rapid and sustained adherence of the graft to the wound surface and the ability to resist shear stress. Sources of grafted tissue can be any known in the art, including exogenous grafts obtained from non-injured tissues in a subject. Exogenous grafts can be, for example, autografts, allografts or xenografts.

Exogenous grafts can likewise be synthetic, e.g. skin substitutes. Synthetic materials suitable for use in grafting include, but are not limited to, silicon, polyurethane, polyvinyl and nylon. Skin substitutes can be any known in the art, including those comprising culture derivatives and cellular or acellular collagen membranes. Culture derived substitutes give rise to bilayer human tissue, for example Apligraf™ comprises epidermal or dermal analogs derived from neonatal foreskin, the host-graft composite of which will become repopulated with cells from the host subject. Commercially available skin substitutes include Biobrane™, composed of silicon, nylon and collagen, TransCyte™, composed of silicon, collagen, fibronectin and glycosaminoglycan, and Integra™, composed of silicon, collagen and glycosaminoglycan. Skin substitutes can be used in applications of permanent and semi-permanent grafting. Preferably, Integra™ is used for permanent grafting.

In grafting tissues, the surface of the graft is aligned to the lesion site through a process known in the art as “approximation.” Approximation of the graft to the lesion site can be carried out according to methods known in the art. For instance, a graft can be placed on top of the lesion site and aligned so that the dye-stained dermal sides are in close approximation. Molecular contact between the graft and the lesion site is achieved by close approximation, which can be performed through pressing and smoothing the dermal-to-dermal composite with several layers of tissue paper, which are then removed without disturbing the graft interface. The approximated graft-lesion site composite is then ready for irradiation.

The photochemical tissue bonding methods described herein can also be used to supplement the use of sutures, e.g., to reinforce sutured anastomosis. Sutures leave a tract behind which can allow for leakage of fluids and organisms. The problem of leakage is especially critical in vascular anastomoses or for any anastomoses of a fluid-containing structure (aorta, ureter, GI tract, eye, etc.) where the fluid or contents inside can leak out through the suture hole. In one embodiment, a wound can be sutured according to general procedures and then treated with the photochemical tissue bonding methods described herein, thereby making the healing wound water tight, and impermeable to bacteria.

In addition, the methods described herein can be used in non surgical wound healing applications, e.g., a “photochemical bandage” can be used for wound healing in addition to, or in place of, a conventional bandage. In one embodiment, a biocompatible substrate, e.g., a conventional bandage material, e.g., a strip of fiber, can be impregnated with the photosensitizer agent described herein, applied to a wound, and photoactivated with a visible light source, e.g., an incandescent, fluorescent or mercury vapor light source, e.g., a xenon arc lamp, or a laser light source. The photochemical bandage can contain another beneficial material for wound healing, e.g., an antibiotic. In some embodiments, the photosensitizer-impregnated bandage, and/or the light source, can be supplied to a subject in a kit, e.g., a kit for use by a health care practitioner, or a kit for household use, which kits can contain instructions for use. The photochemical bandage described herein can be left on the wound, or can be replaced as necessary.

Such a bandage can be used ex-vivo, on a tissue removed from the body, or in situ on a subject, e.g., a human subject. For example, a bandage described herein can be used as an “artificial skin” or covering agent to cover large, oozing surfaces inside or outside the body. Burn patients, for example, could be covered with a photochemical bandage described herein to assist in preventing bacterial infection and to lessen the loss of body fluids and electrolytes through the burned areas.

The methods described herein can also be used to cross-link proteins for use in laboratory applications, e.g., to fix proteins for microscopy; to immobilize antibodies or other protein reagents to a substrate for diagnosis or purification; or to cross link proteins or peptides to a solid matrix for use in chromatographic or immunological applications.

### Kits

The invention also includes kits for use in photochemical tissue bonding. Such kits can be used for laboratory or for clinical applications. Such kits include a photosensitizer agent, e.g., a photosensitizer described herein, and instructions for applying and irradiating the photosensitizer to cross-link at least one protein reagent for laboratory use, or to bond, repair, or heal an animal tissue, e.g., a human tissue, particularly in a human patient. The kits can include a container for storage, e.g., a light-protected and/or refrigerated container for storage of the photosensitizer agent. A photosensitizer included in the kits can be provided in various forms, e.g., in powdered, lyophilized, crystal, or liquid form. Optionally, a kit can include an additional agent for use in a tissue bonding, wound repair, or ocular therapy application, e.g., an antibiotic or a contact lens.

The kits described herein can also include a means to apply the photosensitizer agent to a tissue, for example, a syringe or syringe-like device, a dropper, a powder, an aerosol container, sponge applicator, and/or a bandage material. Kits can further include accessory tools for tissue approximation e.g. clips, standard weights, aspiration apparatus, and compression gauges.

Kits can include instructions for use, e.g., instructions for use in the absence of an exogenously supplied source of cross-linkable substrate, e.g., protein, e.g., fibrin or fibrinogen.

### EXAMPLES

### Example 1

### Assessment of PTB in Repair of Corneal Incisions

PTB can be used to seal or repair a tissue, e.g., a wound, e.g., a corneal wound. This example illustrates the experimental procedure designed to test the efficacy of PTB, as described herein, using mammalian corneas ex vivo. Experiments were performed according to the following procedure.

Rabbit eyes were received on ice (Pel-Freez Biologicals) approximately 17-24 hours after sacrifice and enucleation. The eyes were kept on ice and used the same day. The eye to be studied was mounted on a plastic-covered polystyrene block and fixed in position by needles inserted through the extraocular muscles into the polystyrene. The eye was then placed under a dissecting microscope (Reichert Scientific Instruments, IL) allowing visualization of the treated area during the entire procedure. A 27 G needle was inserted parallel to the iris, 2 mm anterior to the limbus into clear cornea, and positioned above the lens in the anterior chamber. The needle was connected to both a blood pressure transducer (Harvard Apparatus, MA) and a mini-infuser 400 (Bard Harvard) via a T coupler. The pressure transducer consists of a transducer element that is hard wired to an amplifier box and uses a semi-disposable dome with an integral silicone rubber membrane. Pressure inside the dome is transmitted through the membrane to a plastic button whose motion is translated to a voltage. The voltage generated by the transducer amplifier combination is proportional to the lower limit of intraocular pressure (IOP). Signals from the transducer amplifier were recorded using a Macintosh G3 Power book equipped with a PCMICA (DAQCARD - 1200) data acquisition card (National Instruments, TX). Data acquisition was controlled using programs written using the LabView 4 software package (National Instruments, TX). The voltage from the transducer and amplifier was converted to pressure by calibrating with a standing manometer.

Experiments on individual eyes were initiated by increasing the IOP to 30-40 mm Hg, using water infusion at a rate of 1 mL per minute. An incision was made in the cornea, 1 mm from the limbus and parallel to the iris, using a 3.5 mm angled keratome (Becton Dickinson Co.). For each eye the IOP required to produce fluid leakage from the incision (IOP_{L}) was recorded pre- and post- PTB treatment. A photosensitizer, dissolved in phosphate buffer solution (PBS, pH 7.2, Gibco BRL) was applied to the walls of the incision using a Gastight, 50 μl syringe (Hamilton Co.) with a 27 G needle. Confocal fluorescence spectroscopy confirmed the location of photosensitizer, e.g., rose Bengal, on the incision walls and indicated that the photosensitizer penetrated only approximately 100 μM laterally into the wall of the incision.

The photosensitizers, their absorption maxima, and their absorption coefficients at the laser wavelength used in this Example were, e.g., rose bengal (RB), 550 nm, 33000 dm³ mol⁻¹ cm⁻¹ at 514 nm; fluorescein (Fl), 490 nm, 88300 dm³ mol⁻¹ cm⁻¹ at 488 nm; methylene blue (MB), 664 nm, 15600 dm³ mol⁻¹ cm⁻¹ at 661 nm; riboflavin-5-phosphate (R-5-P), 445 nm, 4330 dm³ mol⁻¹ cm⁻¹ at 488 nm; and N-hydroxypyridine-2-(1H)-thione (N-HPT), 314 nm, 2110 dm³ mol⁻¹ cm⁻¹ at 351 nm. The photosensitizers were used as received with the exception of N-HPT which was recrystallized twice from aqueous ethanol before use. The concentrations of the photosensitizers were adjusted so that all the solutions had an absorbance of approximately 1.0 in a path length of 200 μm at the laser irradiation wavelength (with the exception of N-HPT for which the absorption was approximately a factor of 10 lower).

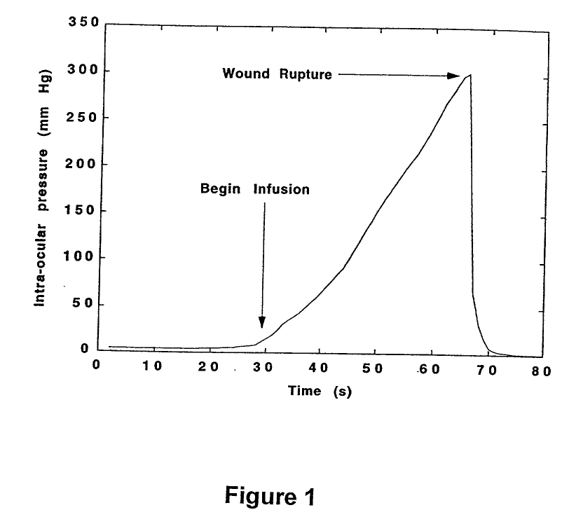

Irradiations employed a continuous wave (CW) argon-ion laser (Innova 100; Coherent, Inc., Palo Alto, Calif.) at 488 nm (for Fl and R-5-P), 514.5 nm (for RB) or 351 nm (for NHPT). An argon-ion-pumped dye laser (CR-599; Coherent) with 4-dicyanomethylene-2-methyl-6-(p-dimethylaminostyryl)-4H-pyran dye (Exciton, Inc., Dayton, Ohio) was used for irradiation at 661 nm (for MB). Laser light was coupled into a 1 mm diameter quartz fiber and a 1 cm diameter spot on the tissue was created by using a combination of 1 and 2 inch focal length, S1-UV grade fused silica, biconvex lenses (Esco Products), mounted in a SM1 series cage assembly (Thorlabs, NJ). The 1 cm diameter circular spot was sufficient to cover the entire incision and the optics were adjusted so that the laser light was incident on the cornea at an angle approximately 45° to the plane of the incision. Dose response curves were obtained by varying the duration of the irradiation at a constant irradiance. In separate experiments the effects of laser irradiance were investigated by comparison of the same delivered dose using different irradiances. The doses used ranged from 124 to 1524 J/cm² and the irradiances used were 0.64, 1.27, 2.55 and 3.86 W/cm². The laser exposure time varied from 33 seconds for the lowest dose using the highest irradiance to 26 minutes, 27 seconds for the highest dose using the lowest irradiance. The IOP_{L} was recorded immediately following treatment. Infusion was started (1 mL per minute) and the IOP increased until a maximum followed by a sharp decrease occurred, corresponding to the opening of the incision and leakage of fluid from the anterior chamber. A typical trace showing the changes in IOP with infusion time is shown in FIG. 1. Five to 10 rabbit eyes were tested for each condition of dose and irradiance.

Control experiments included: (1) irradiation with no photosensitizer application, (2) photosensitizer application only and (3) no photosensitizer or laser irradiation. In the experiments using no photosensitizer, PBS was applied to the incision walls, using the same method as described for the photosensitizers. In control experiments with no laser irradiation the eye was allowed to stand for the same period of time as the laser-treated samples.

### Example 2

### Use of Rose Bengal (RB) in PTB

In the cornea, RB can be used in PTB at a concentration of about 0.5 mM to 5 mM, preferably about 1 mM to 3 mM. The wavelength of irradiation for RB is preferably about 450-600 nm, more preferably about 500 to 560 nm. The dose of irradiation can be from about 0.5 to 2 kJ/cm². The irradiance delivered can be from about 0.2 to 3 W/cm². The duration of irradiation is preferably from about 1 to 10 minutes.

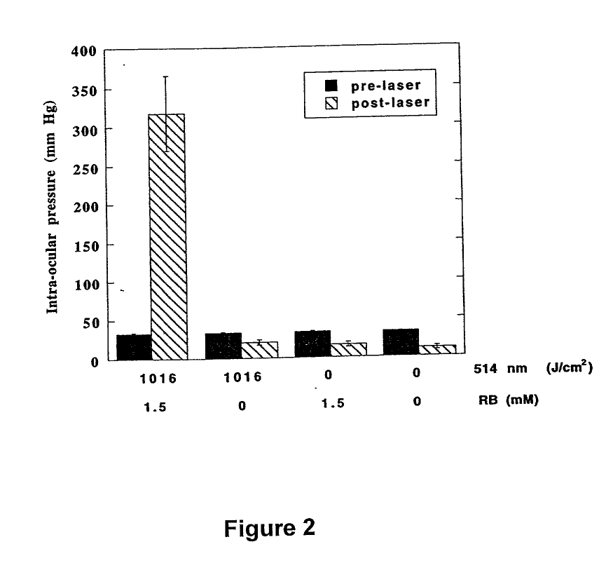

Treatment of incisions with 1.5 mM RB and 514 nm laser light resulted in an increase in post-treatment IOP_{L}, as measured as described in Example 1. Control experiments demonstrated that a significant increase (p<0.005) in the IOP_{L}, following PTB treatment, occurred when both RB and laser irradiation were applied and not by either alone (FIG. 2). The mean IOP_{L} of incisions treated with RB and 514 nm laser light was greater than 300±48 mm Hg, whereas laser irradiation alone or photosensitizer alone produced no significant increase between the pre- and post-treatment IOP_{L} values.

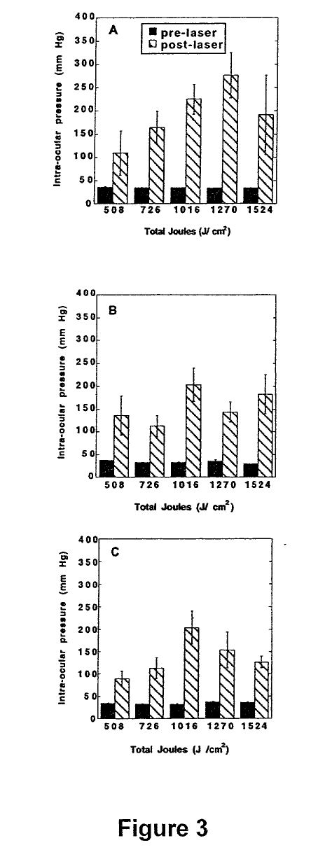

Dose response curves for IOP_{L} are shown in FIG. 3 for RB doses delivered at irradiances of 1.27 (3A), 2.55 (3B) and 3.82 W/cm² (3C). A dose-response relationship was observed at the lowest irradiance (1.27 W/cm²) for doses between 508 and 1270 J/cm² (3A). No significant rise in the IOP_{L} was observed for doses below 508 J/cm² at any irradiance tested. PTB was most efficient at 1270 J/cm² delivered at an irradiance of 1.27 W/cm². All doses delivered at the two lower irradiances (1.27 and 2.55 W/cm²) gave IOP_{L} values greater than 100 mm Hg. Treatment using irradiances of 2.55 and 3.82 W/cm² produced no obvious dose response pattern. In general, for a selected dose the IOP_{L} was lower at higher irradiances. For example, at 1270 J/cm² the mean IOP_{L} values were 274, 150 and 130 mm Hg for the irradiances 1.27 W/cm², 2.55 W/cm² and 3.86 W/cm².

Post-treatment, the eyes were examined for the presence of thermal damage. Tissue shrinkage and deformation around the wound site were taken as signs of thermal damage. Thermal damage to the cornea was not observed at the lowest irradiance tested (1.27 W/cm2). Thermal damage could be observed at doses of 762 to 1524 J/cm2 at the highest irradiance (3.82 W/cm2) and occasionally at 2.55 W/cm2. Thermal effects produced using high irradiances may produce collagen contraction resulting in distortion of the patient's vision.

### Example 3

### Use of Riboflavin-5-Phosphate (R-5-P) in PTB

In the cornea, R-5-P can be applied for PTB at a concentration of about 1 mM to 30 mM, preferably about 10 mM to 20 mM. The wavelength of irradiation for R-5-P is preferably about 400-600 nm, more preferably about 450 to 550 nm. The dose of irradiation can be from about 0.5 to 2 kJ/cm². The irradiance delivered can be from about 0.2 to 3 W/cm². The duration of irradiation is preferably from about 1 to 10 minutes.

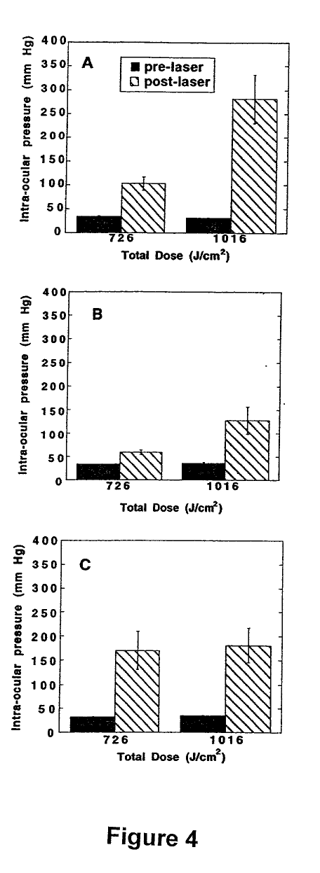

The effect of R-5-P PTB was assessed as described in Example 1. The application of 11 mM R-5-P and irradiation using 488 nm light, at the same irradiances used for RB, and doses of 762 J/cm² and 1016 J/cm², significantly increased the post PTB treatment IOP_{L} value (p<0.05), see FIG. 4. The IOP_{L} values observed using R-5-P are of a similar magnitude to those for RB. However, the IOP_{L} values observed for each dye at the same irradiance and dose were not comparable. Although the treatment produces significant increases in IOP_{L}, no simple pattern between the two dyes is observed.

### Example 4

### Use of N-hydroxypyridine-2-(1H)-thione (N-HTP) in PTB

In the cornea, N-HTP can be applied at a concentration of about 0.5 mM to 10 mM, preferably about 3 mM to 6 mM. The wavelength of irradiation for N-HTP is preferably about 330-400 nm. The dose of irradiation can be from about 0.5 to 2 kJ/cm². The irradiance delivered can be from about 0.2 to 3 W/cm². The duration of irradiation is preferably from about 1 to 10 minutes. A 4.5 mM solution of NHPT was applied to the walls of the incision, as described in Example 1, and irradiated using 351 nm light (0.64 W/cm2) at doses ranging from 127 J/cm2 to 508 J/cm2. Mean IOPL values of 60±23 mm Hg and 126±40 mm Hg were produced when using the doses of 254 J/cm2 and 508 J/cm2 respectively, lower doses than used for the other photosensitizers.

### Example 5

### Use of Methylene Blue (MB) in PTB

MB is a frequently used dye in ophthalmic surgery that has been reported to photosensitize collagen cross-links in rat tail tendon (Ramshaw et al. (1994) Biochim Biophys Acta 1206:225-230). Our previous studies showed that MB and 355 nm light did not produce efficient cross-linking of soluble collagen. MB was therefore used as a control in these ex vivo studies. MB (3 mM) was applied to the walls of the incision, as described in Example 1, and irradiated with 0.64 W/cm² of 661 nm light. Doses of 508/cm², 762 J/cm² and 1016 J/cm² did not increase the post- treatment, IOP_{L}. However, it was observed that MB did not stain the corneal tissue efficiently, which perhaps explains its low efficiency for PTB.

### Example 6

### Assessment of Thermal Contribution to PTB

Laser activated tissue welding has been studied in a variety of tissues (Abergel et al. (1986) J Am Acad Dermatol. 14:810-814; Cilesiz et al., supra; Massicotte et al. (1998) Lasers in Surgery and Medicine 23:18-24; Oz et al. (1990) J Vasc Surg. 11:718-725; Poppas et al. (1996) Lasers in Surgery and Medicine 18:335-344; Poppas et al. (1996) Lasers in Surgery and Medicine 19:360-368; Stewart et al. (1996) Lasers in Surgery and Medicine 19:9-16; Wider et al. (1991) Plastic Reconstr Surg 88:1018-1025). In tissue welding, the laser radiation is used to heat the tissue to temperatures at which collagen denatures and, upon cooling, the collagen molecules intertwine to form a ‘weld’. Additionally, dye-enhanced thermal welding has been investigated (Bass & Treat (1995) Lasers Surg and Med 17:315-349; Chuck et al. (1989) Lasers Surg and Med 9:471-477). In this method the dye selectively absorbs the laser energy and then releases heat to the desired area, reducing peripheral tissue damage. These methods, however, are not appropriate for the cornea due to the potential reduction in visual acuity that would result from the corneal deformation produced by thermal tissue damage. When performing PTB on the cornea, heating must be avoided.

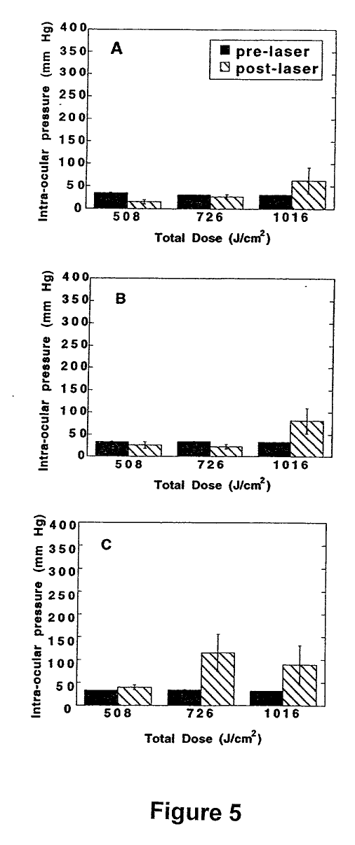

We evaluated the possibility that non-photochemical processes contribute to wound closure by comparing PTB produced by RB with that produced by fluorescein (Fl), a dye with a similar structure but which is not expected to induce protein cross-links. RB and Fl are both xanthene dyes. However, RB is halogenated (4 iodines and 4 chlorines) and the presence of these heavy atoms causes RB to be photochemically active (Lessing et al. (1982) J Mol Struct 84:281-292). Fl has a high quantum yield of fluorescence and lower quantum yield of triplet state formation than RB (Fleming et al. (1977) JACS 99:4306-4311) and will, therefore, produce a lower proportion of active species with the potential to produce collagen cross-links. A solution of 0.6 mM Fl was applied and irradiated using 488 nm laser light at the same range of irradiances used for RB and at doses from 508 J/cm² to 1016 J/cm² (FIG. 5). No increase in IOP_{L} was observed for the incisions treated with the two lowest doses using the two lowest irradiances studied. However, at the highest dose for all irradiances an increase IOP_{L} values was observed with values ranging from 63±30 to 89±42 mm Hg although this is much less efficient than RB (compare FIGS. 3 and 5). These results suggest that PTB is indeed produced by photochemical processes. The IOP_{L} value of 116±40 mm Hg obtained using a dose of 762 J/cm² at 3.82 W/cm² (laser exposure time of 3 min, 10 sec) is considerably higher than any other observed using Fl. The sealing observed at the highest irradiance (3.82 W/cm²) and dose (762 J/cm²) suggests that some other effect is operating, such as a thermal mechanism under these high irradiance conditions.

### Example 7

### PTB Versus Sutures

The IOP_{L} following PTB treatment, as described in Example 1, was compared to that obtained using sutures. Two interrupted radial sutures of black monofilament 10-0 nylon (Ethilon Inc.) were used to close the keratome incision. The sutures were placed in a radial fashion at approximately 90% corneal depth. IOP_{L} values with sutures were approximately 230 mm Hg. This value is similar for the incisions closed with PTB treatment.

### Example 8

### In Vivo PTB

PTB was performed in vivo in New Zealand rabbits to repair two types of corneal wounds.

In group I, 3.5-mm incisions were performed in 20 rabbit (New Zealand White) corneas. Dose and laser irradiance were varied in subgroups of five or more eyes for each condition and appropriate control eyes. Photoactivation was performed with a 514 nm Argon Laser. Wound leak and incisional bursting pressure of the treated and untreated rabbit eyes was determined in vivo, with the animals under anesthetic.

Group I wounds were healed using, e.g., 191 J/cm2, applying 1.5 mM RB. The immediate in vivo bursting pressure was 495±10 mm Hg for PTB treated eyes. Under the same conditions the values of the busting pressure in the control eye varied from 15 to 60 mm Hg. One day after surgery, the bursting pressure was the same for PTB treated eyes and control eyes (approximately 450±125 mmHg). At 14 days, the bursting pressure exceeded 500 mm Hg in both PTB-treated and control eyes.

In Group II, 6-mm Penetrating Keratoplasy (PK) incisions anchored by 4-16 sutures were performed in 16 rabbit corneas. Half of the corneas in each group underwent PTB where 1% Rose Bengal dye was applied to the wound edges followed by laser irradiation at fluence of 191 J/cm2. Photoactivation was performed with a 514 nm Argon Laser and a 532-nm CW Nd: YAG laser. Wound leak and incisional bursting pressure were determined in vivo in the immediate postoperative period. PTB-treated eyes showed an immediate bursting pressure of 410±70 mm Hg for the PTB-treated eyes, compared to 250+150 mm Hg for the control eyes with sutures alone. This result indicate that PTB is useful and effective as a supplement to sutures, as well as on its own.

The results described herein show that PTB is effective to seal, close, or heal a tissue, e.g., a corneal incision, in vivo, in a subject, e.g, an animal, or a human. The presence of a protein, e.g., a protein based sealant, e.g., fibrinogen, is not necessary to obtain a good tissue seal. PTB may be used instead of, or in addition to, other wound healing techniques, e.g., sutures.

### Example 9

### Assessment of PTB in Adhesion of Skin Grafts

Skin grafts and/or skin substitutes are widely used in surgical procedures such as skin transplantation, burn and ulcer wound management and plastic surgery. The primary qualities of successful skin grafts include rapid and sustained adherence to the wound surface and the ability to resist shear stress in order to be void-free and adherent.

To test the ability of PTB to quickly and effectively bond skin grafts to a wound site, an ex vivo model utilizing mini pigs was developed. The use of porcine models in wound healing is well known by those skilled in the art. The similarities between porcine wound healing and that of humans enables one to extrapolate the therapeutic results obtained in a porcine model system to a therapeutic result in humans.

Partial thickness skin grafts of approximately 0.020 inch (corresponding to the thick partial-thickness grafts used in clinical situation) were harvested from mini pigs (2 to 7 months old, weight 15 to 43 kgs) after euthanasia. The grafts were temporarily stored by wrapping the graft around gauze which was soaked in phosphate buffered saline (PBS). The grafts were then immersed in vitrification fluid and cryo-preserved at −80° C. until needed (Fujita T et al. (2000) J Burn Care Rehabil 21:304-9).

The skin grafts were freshly thawed on the day of the experiment and were washed with PBS before being cut into either square biopsies of 1 cm² area or into round biopsies of 0.6 cm diameter. The photosensitizer used was rose bengal (RB) (Sigma), which has the absorption maximum and absorption coefficient of 550 nm and 33,000 dm3/mol/cm at 514 nm, respectively. The RB was dissolved in PBS at concentrations of 0, 0.01, 0.1, and 1% (weight/volume) and kept in darkness before being applied liberally onto the dermal side of the skin grafts for time periods of 30 sec, 1, 2, 5, or 10 minutes, after which time the excess fluid was removed by aspiration and blotting. The round graft was attached to a suture loop while the square graft was secured onto a flat surface with sutures to prepare for the adherence test. The round layer was placed on top of the flat layer with the dye-stained dermal sides in approximation. Excess dye and air at the interface were removed by pressing and rubbing the graft surface over several layers of paper tissue, which was then removed without disturbing the graft interface.

The grafts were irradiated using a continuous-wave (CW) argon-ion laser (Innova 100; Coherent, Palo Alto, Calif.) at 514.5 nm. The approximated grafts were irradiated at a spot size of 0.6 cm diameter, transmitted through a 1 mm diameter quartz fiber. The irradiance of laser applied was 0.56 and 1.68 W/cm² while the dose-dependent response of the laser fluence from 126 to 504 J/cm² was determined. As a result, the exposure time ranged from 2.5 to 15 minutes. During this time the skin grafts were sprayed with PBS at regular intervals in order to prevent dessication.

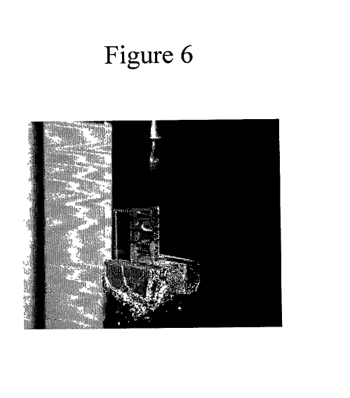

Following the irradiation, the adherence of the skin grafts was tested utilizing a tensiometer (Chartillon) coupled to a force transducer (DFA2, Ametek). The applied force versus the displacement of the force plate was acquired through the transducer and recorded by computer software (Labview 4.0, National Instrument). A force was applied along the direction of the skin grafts at a constant speed of 12.7 mm/min by pulling on the pre-attached suture loop (FIG. 6). Force-deflection plots were obtained, and the total energy needed to separate the skin grafts was calculated and normalized by the volume of the upper (round) graft (energy density: J/cm³) using KaleidaGragh software. This measurement was used to compare the effects of the variations in concentration of RB, dose-response to the change in fluence level as well as differences between the two irradiance levels.

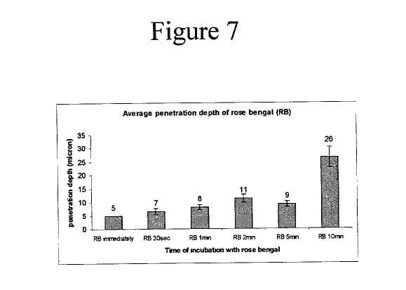

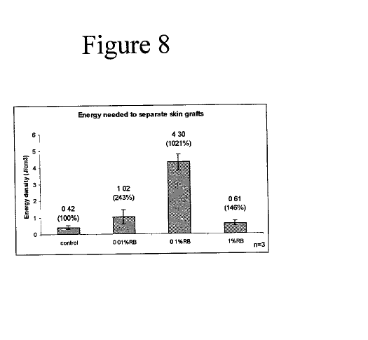

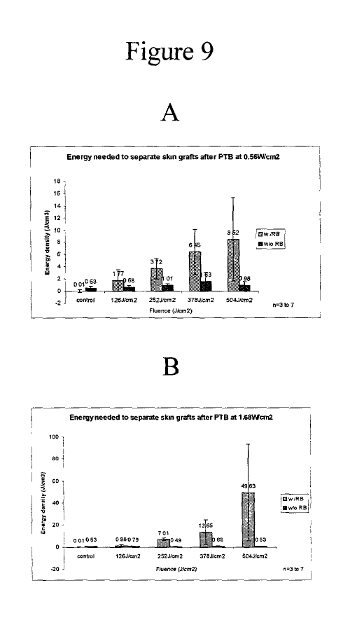

Frozen sections of tissue were analyzed using an eye piece grid on a light microscope to determine the correlation between the time of exposure to RB and the amount of dermal uptake. Exposure times of 5 minutes or less resulted in a dermal uptake having a depth of penetration of approximately 10 microns. When exposure time was increased to 10 minutes, the depth of dermal uptake increased to approximately 25 microns (FIG. 7). The depth of penetration of the dye will be minimized in order to prevent photochemical damage and intrinsic toxicity of the dye if any. Use of RB at a concentration of 0.1% (w/v) provided an optimal increase in adherence levels (FIG. 8). In addition, both irradiation levels (0.56 and 1.68 W/cm²) provided a positive dose-dependent relationship between the laser energy (fluence) and the adherence of the skin grafts (FIGS. 9a and 9b).

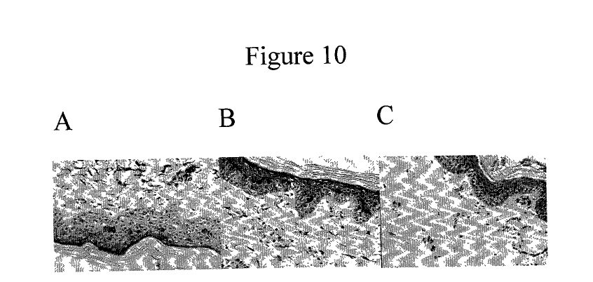

To test the viability of the cells after the irradiation, nicotinamide adenine dinucleotide (NADH-diaphorase) staining was carried out based on the methods described by Heisterkamp J et al. (1999) Lasers Sug Med 25(3):257-62 and Neumann R A et al. (1991) J Am Acad Dermatol 25:991-8. The skin grafts were exposed overnight to an incubation solution consisting of nicotinamide adenine dinuleotide (NADH) and nitroblue tetrazolium chloride (NBT) (Sigma). A water-insoluble blue precipitate formed at the points where NADH-diaphorase activity was present, indicating the viability of the cell. The grafts were washed in PBS and prepared in paraffin sections with a thickness of 5 microns. The sections were counterstained with nuclear fast red to show the cell nuclei. Following irradiation levels of both 0 (control) and 0.56 W/cm², the skin grafts were still viable, as indicated by the dark blue precipitates in the cytoplasm contrasted by the red nuclear counterstain (FIG. 10). However, a higher irradiation level of 1.68 W/cm² resulted in a scarcity of viable cells, and an absence of organization in the collagen bundle.

This example indicates that the utilization of low concentration RB and non-thermal irradiation (e.g. between approximately 0.5 and 1 W/cm²) causes immediate skin graft adherence which maintains graft viability. As a non-thermal bonding method, this type of procedure eliminates the tissue damage caused by thermal bonding methods. The procedures can be varied to incorporate various photosensitizers, appropriate laser light sources, concentrations of RB and amounts of irradiation. This type of treatment is beneficial in humans and would advantageously eliminate the need for staples, sutures, glues, and other adhesives.

A number of embodiments of the invention have been described. Nevertheless, it will be understood that various modifications may be made without departing from the spirit and scope of the invention. Accordingly, other embodiments are within the scope of the following claims.

We claim:

1. A method for grafting tissue, comprising:
identifying a first tissue in need of repair;
contacting the first tissue with a second tissue comprising a tissue graft,
contacting the first tissue, and the second tissue, with at least one photosensitizer agent to form a tissue-photosensitizer complex; and
applying electromagnetic energy to the tissue-photosensitizer complex in a manner effective to produce cross linking in the tissue,
wherein the tissue is not contacted with an exogenous protein or peptide which is cross linked by the application of electromagnetic energy,
thereby creating a tissue seal between the first and second tissue.

2. The method of claim 1, wherein the tissue is skin.

3. The method of claim 1, wherein the at least one photosensitizer agent is selected from the group consisting of Rose Bengal, riboflavin-5-phosphate, and N-hydroxypyridine-2-(1H)-thione.

4. The method of claim 1, wherein the at least one photosensitizer agent is Rose Bengal.

5. The method of claim 1, wherein the contacting steps occurs ex vivo.

6. The method of claim 1, wherein the contacting steps occurs in vivo in a subject.

7. The method of claim 6, wherein the subject is a human.

8. The method of claim 1, wherein the application of electromagnetic energy to the tissue-photosensitizer complex occurs without substantial thermal tissue damage.

9. The method of claim 1, wherein the application of electromagnetic energy to the tissue-photosensitizer complex occurs without more than a 15° C. rise in temperature.

10. The method of claim 1, wherein the application of electromagnetic energy to the tissue-photosensitizer complex occurs without more than a 10° C. rise in temperature.

11. The method of claim 1, wherein the application of electromagnetic energy to the tissue-photosensitizer complex occurs without more than a 3° C. rise in temperature.

12. A method for repairing a skin lesion, comprising:
contacting the skin lesion with a tissue comprising a skin graft,
contacting the skin lesion, and the skin graft, with at least one photosensitizer agent to form a skin tissue-photosensitizer complex; and
applying electromagnetic energy to the skin tissue-photosensitizer complex in a manner effective to elicit the production of a reactive species from the photosensitizer,
wherein the skin lesion and/or graft is not contacted with an exogenous protein or peptide which is cross-linked by the application of electromagnetic energy,
thereby promoting a partial or complete repair of the lesion.

13. The method of claim 12, wherein the skin lesion is caused by a burn, surgical procedure or ulceration.

14. The method of claim 12, wherein repair of the lesion comprises a seal that is resistant to shear stress.

15. The method of claim 12, wherein the electromagnetic energy applied is greater than 100 J/cm².

16. The method of claim 12, wherein the electromagnetic energy is applied at an irradiance less than 1.5 W/cm².

17. The method of claim 12, wherein the electromagnetic energy is applied at an irradiance of about 0.60 W/cm².

18. A method for repairing a skin lesion in vivo in a living subject, comprising:
contacting the skin lesion with a tissue comprising a skin graft; and
contacting the skin lesion, and the skin graft, with Rose Bengal (RB) to form a skin tissue-RB complex; and
applying electromagnetic energy to the skin tissue-RB complex in a manner effective to elicit the production of a reactive oxygen species from the RB,
wherein the skin lesion and/or graft is not contacted with an exogenous protein or peptide which is cross-linked by the application of electromagnetic energy,
thereby promoting a partial or complete repair of the skin lesion.

19. The method of claim 18, wherein the subject is a human.

20. The method of claim 18, wherein the skin lesion is caused by a burn, surgical procedure or ulceration.

21. The method of claim 18, wherein repair of the lesion comprises a seal that is resistant to shear stress.

22. The method of claim 18, wherein the electromagnetic energy is applied at an irradiance less than 1.5 W/cm².

23. The method of claim 18, wherein the electromagnetic energy is applied at an irradiance of about 0.60 W/cm².
